(19) United States
(12) Patent Application Publication
(10) Pub. No.: US 2024/0335304 A1
(43) Pub. Date: Oct. 10, 2024

(54) VALVE SYSTEM FOR HYDRAULIC PROSTHETIC DEVICE

(71) Applicant: College Park Industries, Inc., Warren, MI (US)

(72) Inventors: Jacob Michael Drews, Washington, MI (US); Aaron John Taszreak, China, MI (US); Anna Mae Marlatt, Waterford, MI (US)

(21) Appl. No.: 18/612,157

(22) Filed: Mar. 21, 2024

Related U.S. Application Data
(60) Provisional application No. 63/494,998, filed on Apr. 7, 2023.

(51) Int. Cl. A61F 2/74; A61F 2/64

(52) U.S. Cl. CPC A61F 2/748; A61F 2/64; A61F 2/741

(57) ABSTRACT

A hydraulic valve assembly includes a valve core with one or more valve core ports, the valve core rotatably housed in a valve sleeve having one or more valve sleeve ports. A hydraulic resistance of the hydraulic valve assembly varies approximately linearly with a change in valve position caused by rotating the valve core relative to the valve sleeve to vary an alignment between the one or more valve core ports and one or more valve sleeve ports. The valve assembly optionally includes a motor coupler and a gearmotor, the gearmotor operable to rotate the valve core relative to the valve sleeve via the motor coupler.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims priority benefit of U.S. Provisional Application No. 63/494,998, filed Apr. 7, 2023, the entirety of which is hereby incorporated by reference herein.

### BACKGROUND

### Field

The present disclosure relates generally to a hydraulic valve system for use in a prosthetic device, more specifically, a prosthetic knee.

### Description of the Related Art

Prosthetics are used to replace and restore the functionality of amputated natural body parts. Hydraulics have long been used to control the resistances of prosthetic devices, such as knees, feet, and ankles, via valves that actuated to adjust hydraulic resistance of the prosthetic devices. However, existing hydraulic designs are deficient in that they display an exponential relationship between valve position and hydraulic resistance level during valve position adjustment. Such a large increase in hydraulic resistance level relative to valve position adjustment requires such products to be able to control valve rotation with extreme precision and detect valve position with high accuracy. This is often difficult to achieve and/or results in inaccurate levels of resistance being applied on the hydraulic system.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices, and methods for a hydraulic valve system for a prosthetic device, for example a microprocessor-controlled prosthetic knee.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

In some aspects, the techniques described herein relate to a hydraulic system for a prosthetic device, including: a hydraulic cylinder; and a valve assembly in fluid communication with the hydraulic cylinder, the valve assembly operable to allow flow of a hydraulic fluid to and from the hydraulic cylinder, the valve assembly including: a valve cartridge including: a valve core having one or more valve core ports; and a valve sleeve surrounding the valve core so that the valve core is rotatable relative to the valve sleeve, the valve sleeve including one or more valve sleeve ports, wherein a hydraulic resistance of the valve cartridge varies approximately linearly with a change in valve position of the valve cartridge caused by rotating the valve core relative to the valve sleeve to vary an alignment between the one or more valve core ports and the one or more valve sleeve ports.

In some aspects, the techniques described herein relate to a hydraulic system, wherein the valve core includes a first cylindrical body, and a valve post, the valve post extending distally from the first cylindrical body, the one or more valve core ports arranged circumferentially around the first cylindrical body.

In some aspects, the techniques described herein relate to a hydraulic system, wherein the valve sleeve includes a second cylindrical body, the one or more valve sleeve ports arranged circumferentially around the second cylindrical body.

In some aspects, the techniques described herein relate to a hydraulic system further including a motor.

In some aspects, the techniques described herein relate to a hydraulic system further including a coupler configured to operably couple the motor to a distal end of the valve cartridge, wherein actuation of the motor causes a rotation of the valve core within the valve sleeve.

In some aspects, the techniques described herein relate to a hydraulic system, wherein at least one of the one or more valve sleeve ports is a curved valve sleeve port including a rounded portion and a linear portion.

In some aspects, the techniques described herein relate to a hydraulic system, wherein the linear portion extends at an angle relative to a plane transverse to an axis of the valve cartridge.

In some aspects, the techniques described herein relate to a hydraulic system, wherein the plane is perpendicular to the axis of the valve cartridge.

In some aspects, the techniques described herein relate to a hydraulic system, wherein the angle is equal to or between 2° and 3°.

In some aspects, the techniques described herein relate to a hydraulic system, wherein at least one of the one or more valve sleeve ports is a circular port.

In some aspects, the techniques described herein relate to a hydraulic system, wherein the one or more valve core ports are multiple valve core ports.

In some aspects, the techniques described herein relate to a hydraulic system, wherein at least one of the multiple ports is oblong in shape.

In some aspects, the techniques described herein relate to a hydraulic system, wherein the multiple valve core ports are configured to at least partially overlap with the one or more valve sleeve ports.

In some aspects, the techniques described herein relate to a hydraulic system, further including a valve stop pin coupleable to a coupler and the valve sleeve, the valve stop pin configured to travel within a slot of the coupler between a first stop and a second stop, wherein, at the first stop, the one or more valve core ports and the one or more valve sleeve ports are positioned relative to each other so as to allow a maximum amount of hydraulic fluid flow therethrough at a relatively lower hydraulic resistance, and wherein, at the second stop the one or more valve core ports and the one or more valve sleeve ports are positioned relative to each other so as to allow a minimum amount of hydraulic fluid flow therethrough at a relatively higher hydraulic resistance.

In some aspects, the techniques described herein relate to a hydraulic system, wherein the valve sleeve further includes one or more seals to inhibit leakage of hydraulic fluid.

In some aspects, the techniques described herein relate to a hydraulic system, further including: a motor; and a coupler configured to operably couple the motor to the valve post, wherein actuation of the motor causes a rotation of the coupler and the rotation of the coupler causing a rotation of the valve core relative to the valve sleeve.

In some aspects, the techniques described herein relate to a hydraulic system, wherein the prosthetic device is a prosthetic knee.

In some aspects, the techniques described herein relate to a hydraulic valve assembly including: a valve core having one or more valve core ports; and a valve sleeve surrounding the valve core so that the valve core is rotatable relative to the valve sleeve, the valve sleeve including one or more valve sleeve ports, wherein a hydraulic resistance of the valve assembly varies approximately linearly with a change in valve position of the valve assembly caused by rotating the valve core relative to the valve sleeve to vary an alignment between the one or more valve core ports and one or more valve sleeve ports.

In some aspects, the techniques described herein relate to a hydraulic valve assembly, wherein the valve core includes a first cylindrical body, and a valve post, the valve post extending distally from the first cylindrical body, the one or more valve core ports arranged circumferentially around the first cylindrical body.

In some aspects, the techniques described herein relate to a hydraulic valve assembly, wherein the valve sleeve includes a second cylindrical body, the one or more valve sleeve ports arranged circumferentially around the second cylindrical body.

In some aspects, the techniques described herein relate to a hydraulic valve assembly, wherein at least one of the one or more valve sleeve ports is a curved valve sleeve port including a rounded portion and a linear portion.

In some aspects, the techniques described herein relate to a hydraulic valve assembly, wherein the linear portion extends at an angle relative to a plane transverse to an axis of the valve assembly.

In some aspects, the techniques described herein relate to a hydraulic valve assembly, wherein the plane is perpendicular the axis of the valve assembly.

In some aspects, the techniques described herein relate to a hydraulic valve assembly, wherein the angle is equal to or between 2° and 3°.

In some aspects, the techniques described herein relate to a hydraulic valve assembly, wherein at least one of the one or more valve sleeve ports is a circular port.

In some aspects, the techniques described herein relate to a hydraulic valve assembly, wherein the one or more valve core ports are multiple valve core ports.

In some aspects, the techniques described herein relate to a hydraulic valve assembly, wherein at least one of the multiple valve core ports is oblong in shape.

In some aspects, the techniques described herein relate to a hydraulic valve assembly, wherein the multiple valve core ports are configured to at least partially overlap with the one or more valve sleeve ports.

In some aspects, the techniques described herein relate to a hydraulic valve assembly, wherein the valve sleeve further includes one or more seals to inhibit leakages of hydraulic fluid.

In some aspects, the techniques described herein relate to a device for actuation of a prosthetic, including: a hydraulic cylinder; a piston movably coupled to the hydraulic cylinder and configured to linearly move within the hydraulic cylinder; a motorized valve system, the motorized valve system including: a motor, and a valve assembly, including: a valve core having one or more valve core ports; and a valve sleeve surrounding the valve core so that the valve core is rotatable relative to the valve sleeve, the valve sleeve including one or more valve sleeve ports, wherein a hydraulic resistance of the valve assembly varies approximately linearly with a change in valve position of the valve assembly caused by rotating the valve core relative to the valve sleeve to vary an alignment between the one or more valve core ports and one or more valve sleeve ports.

In some aspects, the techniques described herein relate to a device, wherein the valve core includes a first cylindrical body, and a valve post, the valve post extending distally from the first cylindrical body, the one or more valve core ports arranged circumferentially around the first cylindrical body.

In some aspects, the techniques described herein relate to a device, wherein the valve sleeve includes a second cylindrical body, the one or more valve sleeve ports arranged circumferentially around the second cylindrical body.

In some aspects, the techniques described herein relate to a device, further including a coupler configured to operably couple the motor to a distal end of the valve assembly, wherein actuation of the motor causes a rotation of the valve core within the valve sleeve.

In some aspects, the techniques described herein relate to a device, wherein at least one of the one or more valve sleeve ports is a curved valve sleeve port including a rounded portion and a linear portion.

In some aspects, the techniques described herein relate to a device, wherein the linear portion extends at an angle relative to a plane transverse to an axis of the valve assembly.

In some aspects, the techniques described herein relate to a device, wherein the plane is perpendicular the axis of the valve assembly.

In some aspects, the techniques described herein relate to a device, wherein the angle is equal to or between 2° and 3°.

In some aspects, the techniques described herein relate to a device, wherein at least one of the one or more valve sleeve ports is a circular port.

In some aspects, the techniques described herein relate to a device, wherein the one or more valve core ports are multiple valve core ports.

In some aspects, the techniques described herein relate to a device, wherein at least one of the multiple valve core ports is oblong in shape.

In some aspects, the techniques described herein relate to a device, wherein the multiple valve core ports are configured to at least partially overlap with the one or more valve sleeve ports.

In some aspects, the techniques described herein relate to a device, further including a valve stop pin couplable to a coupler and the valve sleeve, the valve stop pin configured to travel within a slot of the coupler between a first stop where the one or more valve core ports and the one or more valve sleeve ports are positioned relative to each other so as to allow a maximum amount of hydraulic fluid flow therethrough at a relatively lower hydraulic resistance, and a second stop where the one or more valve core ports and the one or more valve sleeve ports are positioned relative to each other so as to allow a minimum amount of hydraulic fluid flow therethrough at a relatively higher hydraulic resistance.

In some aspects, the techniques described herein relate to a device, wherein the valve sleeve further includes one or more seals to inhibit leakage of hydraulic fluid.

In some aspects, the techniques described herein relate to a device, wherein the prosthetic is a prosthetic knee.

In accordance with one aspect of the disclosure, a hydraulic valve system for use in a prosthetic device is provided. The hydraulic valve system, in operation, provides an approximately linear relationship between hydraulic resistance and valve position, advantageously allowing for more precise control of hydraulic resistance as the valve position is changed (e.g., as the valve is rotated) and less stringent valve positioning requirements to achieve a desired hydraulic resistance level. In one example, the prosthetic device is a prosthetic knee.

In accordance with one aspect of the disclosure, a hydraulic system for a prosthetic device is provided. The hydraulic system includes a hydraulic cylinder and a valve assembly in fluid communication with the hydraulic cylinder. The valve assembly is operable to allow flow of a hydraulic fluid to and from the hydraulic cylinder. The valve assembly includes a valve cartridge including a valve core having one or more valve core ports, and a valve sleeve surrounding the valve core so that the valve core is rotatable relative to the valve sleeve. The valve sleeve includes one or more valve sleeve ports. A hydraulic resistance of the valve cartridge varies approximately linearly with a change in valve position of the valve cartridge caused by rotating the valve core relative to the valve sleeve to vary an alignment between the one or more valve core ports and the one or more valve sleeve ports.

In accordance with another aspect of the disclosure, a hydraulic valve assembly is provided. The hydraulic valve assembly includes a valve core having one or more valve core ports, and a valve sleeve surrounding the valve core so that the valve core is rotatable relative to the valve sleeve. The valve sleeve includes one or more valve sleeve ports. A hydraulic resistance of the valve assembly varies approximately linearly with a change in valve position of the valve assembly caused by rotating the valve core relative to the valve sleeve to vary an alignment between the one or more valve core ports and one or more valve sleeve ports.

In accordance with another aspect of the disclosure, a device for actuation of a prosthetic is provided. The device includes a hydraulic cylinder, a piston movably coupled to the hydraulic cylinder and configured to linearly move within the hydraulic cylinder, and a motorized valve system. The motorized valve system includes a motor and a valve assembly. The valve assembly includes a valve core having one or more valve core ports, and a valve sleeve surrounding the valve core so that the valve core is rotatable relative to the valve sleeve. The valve sleeve includes one or more valve sleeve ports. A hydraulic resistance of the valve assembly varies approximately linearly with a change in valve position of the valve assembly caused by rotating the valve core relative to the valve sleeve to vary an alignment between the one or more valve core ports and one or more valve sleeve ports.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

The following drawings are for illustrative purposes only and show non-limiting embodiments. Features from different figures may be combined in several embodiments.

FIG. 1A is a side view of an example of a prosthetic knee containing a hydraulic valve system.

FIG. 1B is a front view of an example of a hydraulic valve system embedded in a hydraulic cylinder.

FIG. 2 is a schematic side view of an example of the hydraulic valve system in FIGS. 1A-1B.

FIG. 3 is a schematic side view of an example valve core.

FIG. 4 is schematic side view of an example valve sleeve.

FIG. 5 is a schematic side view of the valve cartridge of FIG. 4 and an example motor coupling.

FIG. 6 is a graph showing the relationship between valve position and resistance at various speeds of hydraulic cylinder actuation.

### DETAILED DESCRIPTION

The following detailed description is directed to certain specific embodiments of prosthetic devices and methods. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. The embodiments, examples of which are illustrated in the accompanying drawings, are set forth in detail below. Wherever possible, the same reference numbers are used throughout the drawings to refer to the same or like parts.

Microprocessor-controlled hydraulic prosthetic knees are an ideal solution for controlling the rotation of the prosthetic knee joint for above knee amputees. These systems employ one or more microprocessors and various sensors that detect, respond, and react to the bending of the knee joint by modifying the hydraulic resistance in both flexion and extension directions. Such modification of hydraulic resistance can be achieved by the use of a valve-system coupled with a hydraulic cylinder. While the following disclosure describes a hydraulic valve system with respect to a microprocessor-controlled hydraulic prosthetic knee, one of skill in the art will understand that the valve system may be suitable for use in other types of prosthetics (e.g., prosthetic feet, prosthetic ankles, etc.) and prosthetics that are not microprocessor-controlled (e.g., prosthetics where hydraulic resistance is manually adjustable by a user or prosthetist by manually changing a valve position of a hydraulic valve system).

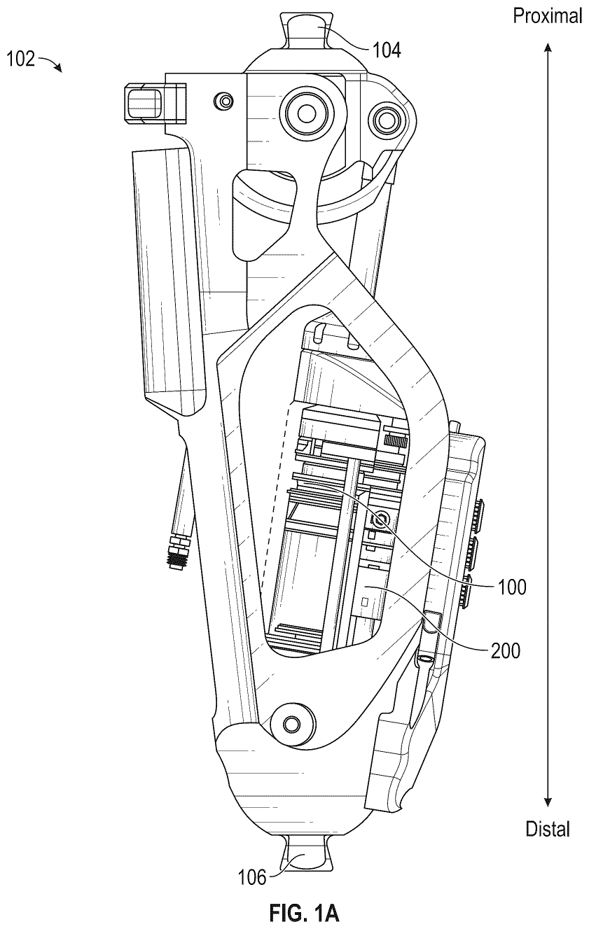

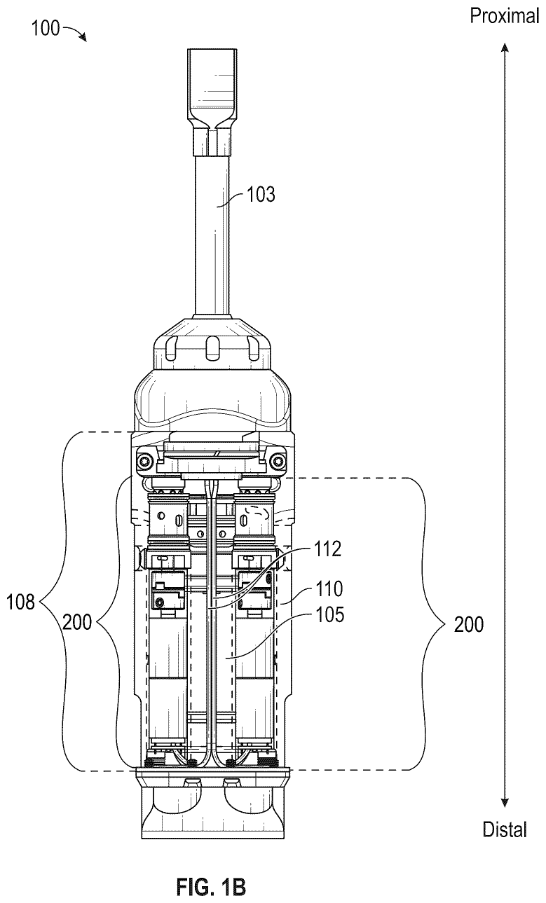

FIG. 1A is a side view of an example of a prosthetic knee 102 (e.g. a micro-processor controlled prosthetic knee) containing the valve system 200 described herein. The prosthetic knee 102 includes a hydraulic cylinder 100 in which the valve system 200 is housed, as described further below with respect to FIG. 1B. A piston (not shown) can move within a chamber 105 of the hydraulic cylinder 100 (e.g., to adjust a position of the prosthetic knee). The proximal end of the prosthetic knee 102 may include a proximal connector 104 (e.g., pyramid connector) for connecting the prosthetic knee 102 to an upper leg prosthetic device (e.g., a socket worn by an amputee over their upper leg). The distal end of the prosthetic knee 102 may include a distal connector 106 (e.g., pyramid connector) for connecting the prosthetic knee to a lower leg prosthetic device (e.g., a pylon that couples to a prosthetic foot, a prosthetic foot, etc.).

FIG. 1B is a front view of an example of the valve system 200 embedded in a hydraulic cylinder 100. The cylinder body 108 may define one or more hollow shafts or bores (not shown) each housing a valve system 200. The cylinder body 108 may have a circumferential cylinder wall 110. The valve system 200 may be in fluid communication with the chamber 105 of the hydraulic cylinder 100 in which the piston 112 (that is attached to a distal end of the piston rod 103 travels. In the example shown in FIG. 1B, the hydraulic cylinder 100 houses two valve systems 200—one operated to control resistance in an extension movement of the prosthetic device and the other to control resistance in a flexion movement of the prosthetic device. In other examples, the hydraulic cylinder 100 may house only one valve system 200 that is operated to control resistance in both flexion and extension movements.

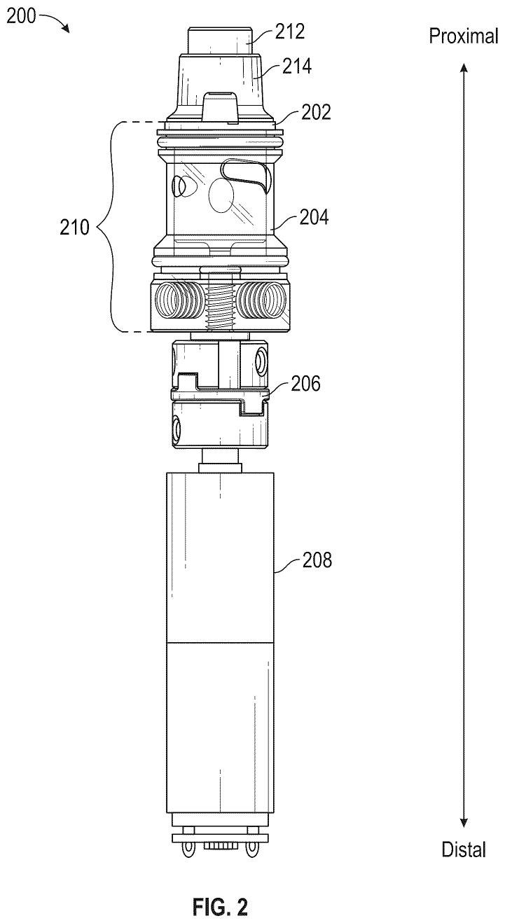

FIG. 2 is a side view of an example of the valve system 200 in FIGS. 1A-1B. In some embodiments, the valve system 200 may include, at its proximal end, a valve cartridge 210 including a valve core 202 surrounded by a valve sleeve 204. The dimensions of the valve core 202 and the valve sleeve 204 allow the valve core 202 to fit inside the valve sleeve 204 while leaving enough space between the outer surface of the valve core 202 and the inner surface of the valve sleeve 204 to allow the valve core 202 to rotate within the valve sleeve 204. In some embodiments, the valve core 202 may remain fixed and the valve sleeve 204 may be rotated relative to the valve core 202. The valve core 202 and valve sleeve 204 may be constructed from any suitable material (e.g., steel). The valve core 202 and valve sleeve 204 may have any appropriate dimensions. In some embodiments, the valve system 200 may include a gearmotor 208 operably connected to the distal end of the valve cartridge 210 by a motor coupler 206. The distal end of the motor coupler 206 may be attached the proximal end of the gearmotor 208 such that the motor coupler 206 may transfer motion (e.g., rotational motion) from the gearmotor 208 to the valve core 202 (e.g., to rotate the valve core 202 relative to the valve sleeve 204). In some embodiments, the valve system 200 may include a magnet 212 magnetically coupled to the distal facing surface of the valve core 202. The magnet 212 may be used to provide position of the valve core 202 to a Hall effect sensor (not pictured). The magnet 212 is diametrically polarized and housed in a magnet receptacle 214 coupled to the valve core 202 (e.g., by press-fit). Rotation of the valve core 202 within the valve sleeve 204 will thus cause the magnet receptacle 214 and magnet 212 to rotate. The angular rotation is detected by the Hall effect sensor (not shown), which advantageously allows for controlling the positioning of the valve core 202 and thus the resistance provided. The magnet receptacle 214 may be constructed from a plastic material to advantageously prevent magnetic properties of any other components of the valve system 200 from interfering and/or distorting the magnetic field of the magnet 212, allowing for accurate position detection by the Hall effect sensor (not shown).

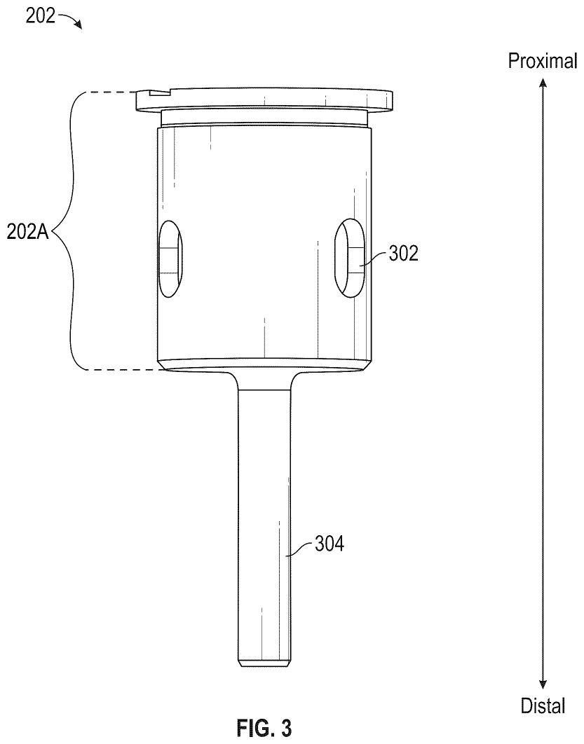

FIG. 3 is a side view of an example valve core 202 of the valve system 200. The proximal end of the valve core 202 may be a hollow cylindrical structure 202A having one or more (e.g., one, two, three, four, etc.) openings or ports 302 (e.g., distributed evenly) around the circumference of the hollow cylindrical structure 202A. The one or more valve core ports 302 may allow for hydraulic fluid flow therethrough (e.g., between the valve core 202 and the hydraulic cylinder 100). In some embodiments, each valve core port 302 may have an ovular or oblong shape, although other shapes may also be suitable (e.g., circular, square, etc.). The proximal facing surface of the valve core 202 may include a magnet receptacle (not shown) to facilitate coupling of the magnet 212 to the valve core 202. The distal end of the valve core 202 may include a valve post 304. The distal end of the valve core post 304 may be operably coupled to the proximal end of the motor coupler 206 and the rotational motion is transferred from the gearmotor 208 by the motor coupler 206 to the valve core 302 via the valve post 304, causing rotation of the valve core 202 within the valve sleeve 204.

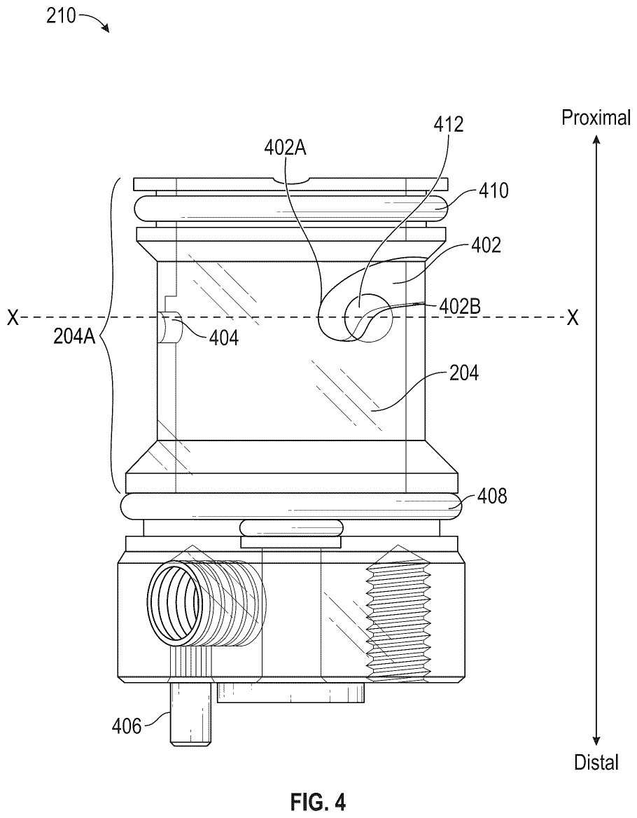

FIG. 4 is a side view of an example valve sleeve 204. The proximal end of the valve sleeve 204 may be a hollow cylindrical structure 204A similar to the cylindrical structure 202A of the proximal end of the valve core 202, but the circumference of the cylindrical structure 204A of the valve sleeve 204 may be larger than the circumference of the cylindrical structure 202A of the valve core 202 such that the valve core 202 may fit inside of the valve sleeve 204 as discussed above with respect to FIG. 2. The proximal end of valve sleeve 204 may include one or more (e.g., one, two, three, four, etc.) openings or ports (e.g., distributed evenly) around the circumference of the cylindrical structure 204A. The number of ports in the valve sleeve 204 can be equal to the number of ports in the valve core 202. One of the ports in the valve sleeve 204 may be a curved port 402 with a rounded portion 402A and a linear portion 402B that extends at angle (e.g., 2° or 3°) relative to a horizontal plane or line (e.g., see line X-X in FIG. 4). Advantageously, the manner in which the curved port 402 overlaps with the ports 302 of the valve core 202 during operation of the valve system 200 yields an approximately linear relationship between the valve position (e.g., rotation of the valve core 202 relative to the valve sleeve 204) and a hydraulic resistance provided by the valve system 200. In other examples, the port 402 can have other suitable shapes that yield an approximately linear relationship between the valve position and the hydraulic resistance provided by the valve system 200. The remaining port(s) of the valve sleeve 204 may be circular ports 404 or have another suitable shape.

The curved port 402 and round port(s) 404 may be (at least partially) disposed on the same lateral plane X-X as the valve core ports 302 when the valve core 202 is disposed within the valve sleeve 204. The amount and/or rate of hydraulic fluid flow between the valve cartridge 210 and the hydraulic cylinder 100 may be controlled by the rotational position of the valve core 202 relative to the valve sleeve 204. When the valve core 202 is rotated (e.g., manually, or electronically via the gearmotor 208 and the motor coupler 206) to a position where the valve core ports 302 are aligned with the valve sleeve ports 402, 404, an opening 412 is formed by the aligned ports 302, 402, 404 that allows hydraulic fluid flow in and out of the valve cartridge 210. When the valve core 202 is rotated (e.g., manually, or electronically via the gearmotor 208 and the motor coupler 206) to a position where the valve core ports 302 are not aligned with the ports 402, 404 of the valve sleeve 204, the wall (e.g., solid portion) of the valve sleeve 204 will slow or inhibit (e.g., prevent) hydraulic fluid from flowing into and out of the valve cartridge 210, thereby increasing the hydraulic resistance of the valve system 200.

The geometry of the curved port 402 of the valve sleeve 204 relative to the ovular or oblong valve core ports 302 (e.g., and the overlap between the curved port 402 and the valve core ports 302 as the valve core 202 is rotated relative to the valve sleeve 204) advantageously provides or results in an approximately linear relationship between the hydraulic resistance and the rotational position of the valve core 202 relative to the valve sleeve 204 (e.g., a rotational position of the valve cartridge 210). When a valve core port 302 is in line with the rounded portion 402A of the curved port 402, more hydraulic fluid may flow into and out of the valve cartridge 210 through the opening 412 and the valve system 200 can have a relatively lower hydraulic resistance. As the valve core 202 is rotated and passes relative to the linear portion 402B of the curved port 402, the amount of hydraulic fluid flow through the valve cartridge 210 (e.g., the amount that passes through the opening 412 formed by the overlapping portions of the valve core port 302 and the curved portion 402 of the valve sleeve 204) is gradually decreased until the valve core port 302 is no longer aligned with the curved port 402, at which point hydraulic fluid flow is blocked by the solid portion of the valve sleeve 204. The valve sleeve 204 may further include a proximal seal 410 (e.g., O-ring) and a distal seal 408 (e.g., O-ring) to inhibit (e.g., prevent) unwanted hydraulic fluid flow around the valve cartridge 210 (e.g., so that hydraulic fluid only passes through the valve cartridge 210 via the valve core ports 302 and the ports 402, 404 of the valve sleeve 204).

Having a linear relationship between valve rotation and hydraulic resistance advantageously allows for more precise control over the change in resistance of the prosthetic joint (e.g. prosthetic knee 102) as the valve core 202 is rotated relative to the valve sleeve 204 (e.g., to change the valve position). Additionally, control of the gearmotor 208 and the position of the valve core 202 relative to the valve sleeve 204 to achieve the desired hydraulic resistance advantageously does not require a high level (e.g., extreme) accuracy. This allows for more leniency in the valve position requirements needed for the valve cartridge 210 design to achieve a desired resistance, which results in lower manufacturing costs, higher manufacturing efficiency and a more effective valve design that may require less precision to operate as desired. This in turn allows for increased manufacturing production viability as the variability in supplied resistance with valve cartridge 210 position will be reduced. Further, recalibration of the valve cartridge 210 may advantageously not be needed as the change in resistance from one valve position to the next is reduced greatly (e.g., due to the approximately linear relationship between valve position and hydraulic resistance level) when compared to systems that have an exponential relationship (described above). Also, any backlash that may be experienced by the valve system 200 will advantageously have a reduced effect on the resistance provided (again due to the approximately linear relationship between valve position and hydraulic resistance level). If the valve position is missed (e.g., not correctly set, such as by the gearmotor 208), because the relationship between valve position and hydraulic resistance level is linear and not exponential, the impact on the user will be minimal and will not be detectable, for example, during a regular walking motion.

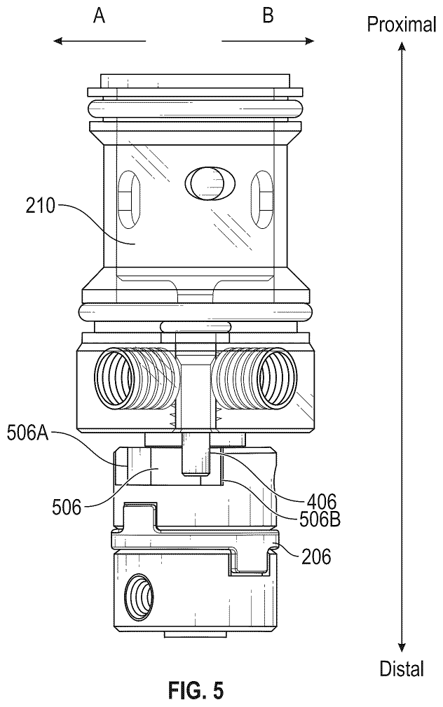

The valve sleeve 204 may have a stop pin 406. The stop pin 406 may control the range of rotational motion of the valve core 202 relative to the valve sleeve 204 as further described with respect to FIG. 5 below. The stop pin 406 can at least partially extend into an opening or bore in a distal end of the valve sleeve 204, a distal end of the stop pin 406 protruding from the distal end of the valve sleeve 204 and extending into a slot or groove 506 in the motor coupler 206.

FIG. 5 is a side view of the valve cartridge 210 of FIG. 5 and an example motor coupler 206. As described above, rotational motion from the gearmotor 208 is transferred to the motor coupler 206 which is connected to the valve post 304 to cause rotation of the valve core 202 (e.g., via rotation of the motor coupler 206). The valve cartridge 210 may be rotated between a fully open configuration (e.g., when a valve core port 302 is aligned with one of the valve sleeve 204 ports 402, 404 allowing a maximum amount hydraulic fluid flow through the valve cartridge 210 at a lower hydraulic resistance, such as a minimum hydraulic resistance) and a fully closed configuration (e.g., where hydraulic fluid flow through the valve cartridge 210 is smaller, for example at a minimum amount, and at a relatively higher hydraulic resistance, such as a maximum hydraulic resistance). The proximal facing side of the motor coupler 206 has a slot or groove 506 that receives the distal end of the stop pin 406 during rotation. When the valve cartridge 210 is being rotated to the fully open configuration, the motor coupler 206 will rotate in the direction indicated by arrow “B”. When the valve cartridge 210 reaches the fully open configuration, the stop pin 406 will contact a first end 506A of the slot or groove 506 and inhibit (e.g., prevent) further rotation of the valve core 202 from occurring. When the valve cartridge 210 is being rotated to the fully closed configuration, the motor coupler 206 will rotate in the direction indicated by arrow “A”. When the valve cartridge 210 reaches the fully closed configuration, the stop pin 406 will contact a second end 506B of the slot or groove 506 and inhibit (e.g., prevent) further rotation of the valve core 202 from occurring.

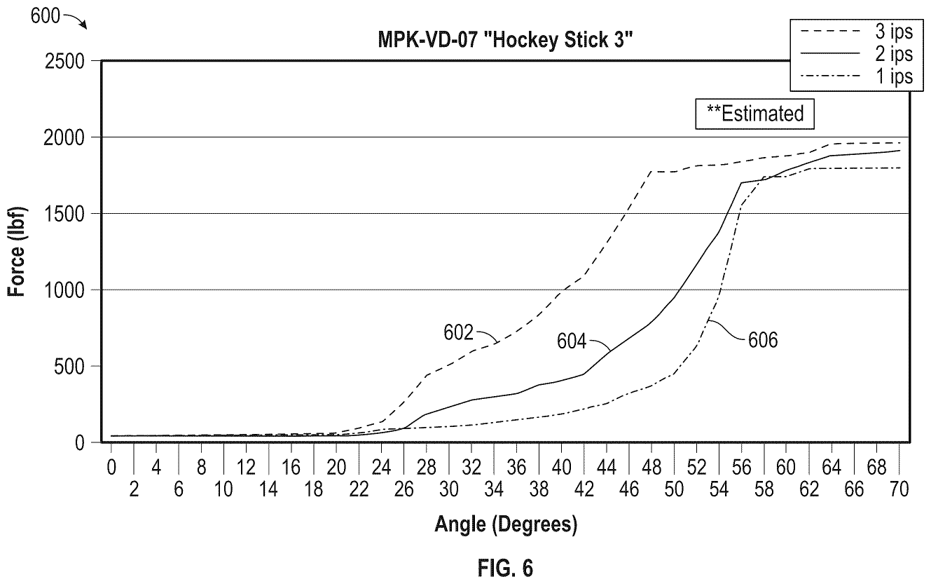

FIG. 6 is a graph 600 showing the approximately linear relationship between valve position and resistance at various speeds of rotation in the valve system 200 described herein. The graph 600 shows the relationship between valve position and valve hydraulic resistance as the valve system 200 is rotated between the fully open configuration and the fully closed configuration. Line plot 602 shows the relationship between valve position and valve hydraulic resistance when the piston 112 of the hydraulic cylinder 100 is actuated at a rate of 3 inches/sec. Line plot 604 shows the relationship between valve position and valve hydraulic resistance when the piston 112 of the hydraulic cylinder 100 is actuated at a rate of 2 inches/sec. Line plot 606 shows the relationship between valve position and valve hydraulic resistance when the piston 112 of the hydraulic cylinder 100 is actuated at a rate of 1 inches/sec. As shown in FIG. 6, while the valve core 202 is rotated relative to the valve sleeve 204 over approximately the first 20 degrees, the hydraulic resistance remains low (e.g., the valve is in a fully open position to freely allow fluid flow therethrough), and between approximately 20 degrees and approximately 48 degrees (for line plot 602) the hydraulic resistance increases approximately linearly relative to valve position. At a rotational position of greater than 48 degrees, the hydraulic resistance continues to increase (e.g., to a point where the valve approximates a fully closed position and inhibits or prevents hydraulic fluid flow therethrough). Line plot 604 exhibits a similar approximately linear profiles but offset to the right in the graph 600.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

Conditional language, such as “may,” “could,” “might,” “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A hydraulic system for a prosthetic device, comprising:
a hydraulic cylinder; and
a valve assembly in fluid communication with the hydraulic cylinder, the valve assembly operable to allow flow of a hydraulic fluid to and from the hydraulic cylinder, the valve assembly comprising:
a valve cartridge comprising:
a valve core having one or more valve core ports; and
a valve sleeve surrounding the valve core so that the valve core is rotatable relative to the valve sleeve, the valve sleeve comprising one or more valve sleeve ports,
wherein a hydraulic resistance of the valve cartridge varies approximately linearly with a change in valve position of the valve cartridge caused by rotating the valve core relative to the valve sleeve to vary an alignment between the one or more valve core ports and the one or more valve sleeve ports.

2. The hydraulic system of claim 1, wherein the valve core comprises a first cylindrical body, and a valve post, the valve post extending distally from the first cylindrical body, the one or more valve core ports arranged circumferentially around the first cylindrical body.

3. The hydraulic system of claim 1, wherein the valve sleeve comprises a second cylindrical body, the one or more valve sleeve ports arranged circumferentially around the second cylindrical body.

4. The hydraulic system of claim 1, wherein at least one of the one or more valve sleeve ports is a curved valve sleeve port comprising a rounded portion and a linear portion.

5. The hydraulic system of claim 1, further comprising a valve stop pin coupleable to a coupler and the valve sleeve, the valve stop pin configured to travel within a slot of the coupler between a first stop and a second stop, wherein, at the first stop, the one or more valve core ports and the one or more valve sleeve ports are positioned relative to each other so as to allow a maximum amount of hydraulic fluid flow therethrough at a relatively lower hydraulic resistance, and wherein, at the second stop the one or more valve core ports and the one or more valve sleeve ports are positioned relative to each other so as to allow a minimum amount of hydraulic fluid flow therethrough at a relatively higher hydraulic resistance.

6. A hydraulic valve assembly comprising:
a valve core having one or more valve core ports; and
a valve sleeve surrounding the valve core so that the valve core is rotatable relative to the valve sleeve, the valve sleeve comprising one or more valve sleeve ports,
wherein a hydraulic resistance of the valve assembly varies approximately linearly with a change in valve position of the valve assembly caused by rotating the valve core relative to the valve sleeve to vary an alignment between the one or more valve core ports and one or more valve sleeve ports.

7. The hydraulic valve assembly of claim 6, wherein at least one of the one or more valve sleeve ports is a curved valve sleeve port comprising a rounded portion and a linear portion.

8. The hydraulic valve assembly of claim 7, wherein the linear portion extends at an angle relative to a plane transverse to an axis of the valve assembly.

9. The hydraulic valve assembly of claim 8, wherein the plane is perpendicular the axis of the valve assembly.

10. The hydraulic valve assembly of claim 8, wherein the angle is equal to or between 2° and 3°.

11. The hydraulic valve assembly of claim 6, wherein at least one of the one or more valve sleeve ports is a circular port.

12. The hydraulic valve assembly of claim 6, wherein the one or more valve core ports are multiple valve core ports.

13. The hydraulic valve assembly of claim 12, wherein at least one of the multiple valve core ports is oblong in shape.

14. The hydraulic valve assembly of claim 13, wherein the multiple valve core ports are configured to at least partially overlap with the one or more valve sleeve ports.

15. A device for actuation of a prosthetic, comprising:
a hydraulic cylinder;
a piston movably coupled to the hydraulic cylinder and configured to linearly move within the hydraulic cylinder;
a motorized valve system, the motorized valve system comprising:
a motor, and
a valve assembly, comprising:
a valve core having one or more valve core ports; and
a valve sleeve surrounding the valve core so that the valve core is rotatable relative to the valve sleeve, the valve sleeve comprising one or more valve sleeve ports,
wherein a hydraulic resistance of the valve assembly varies approximately linearly with a change in valve position of the valve assembly caused by rotating the valve core relative to the valve sleeve to vary an alignment between the one or more valve core ports and one or more valve sleeve ports.

16. The device of claim 15, wherein the valve core comprises a first cylindrical body, and a valve post, the valve post extending distally from the first cylindrical body, the one or more valve core ports arranged circumferentially around the first cylindrical body.

17. The device of claim 15, wherein the valve sleeve comprises a second cylindrical body, the one or more valve sleeve ports arranged circumferentially around the second cylindrical body.

18. The device of claim 15, further comprising a coupler configured to operably couple the motor to a distal end of the valve assembly, wherein actuation of the motor causes a rotation of the valve core within the valve sleeve.

19. The device of claim 15, wherein the valve sleeve further comprises one or more seals to inhibit leakage of hydraulic fluid.

20. The device of claim 15, wherein the prosthetic is a prosthetic knee.
